(19) United States
(12) Patent Application Publication
(10) Pub. No.: US 2025/0352181 A1
(43) Pub. Date: Nov. 20, 2025

(54) ULTRASONIC DIAGNOSTIC APPARATUS, IMAGE DISPLAY METHOD, AND RECORDING MEDIUM

(71) Applicant: Konica Minolta, Inc., Tokyo (JP)

(72) Inventors: Kaoru Okada, Tokyo (JP); Masahiko Kadokura, Ogaki-shi (JP); Koji Oura, Yokohama-shi (JP); Kazuya Takagi, Tokyo (JP); Takashi Mizuno, Tokyo (JP)

(73) Assignee: Konica Minolta, Inc., Tokyo (JP)

(21) Appl. No.: 19/208,047

(22) Filed: May 14, 2025

(30) Foreign Application Priority Data
May 16, 2024 (JP) 2024-079925

(51) Int. Cl. A61B 8/00; A61B 90/00

(52) U.S. Cl. CPC A61B 8/463; A61B 8/4263; A61B 8/5207; A61B 90/361

(57) ABSTRACT

Disclosed is an ultrasonic diagnostic apparatus including: an ultrasound image generator that generates ultrasound image data from reception signals of an ultrasound probe that transmits and receives ultrasound waves to and from a subject; an optical image capturer that generates optical image data by optically imaging puncture into the subject using a puncture needle; and a hardware processor that simultaneously displays an ultrasound image of the ultrasound image data and a reference image based on the optical image data, detects a position of a user in an optical image of the optical image data, and sets an orientation of the reference image to be displayed so that the detected position of the user is located on a lower side.

### BACKGROUND OF THE INVENTION

### Technical Field

The present invention relates to an ultrasonic diagnostic apparatus, an image display method, and a recording medium.

### Description of Related Art

There has been conventionally known an ultrasonic diagnostic apparatus that emits ultrasound with an ultrasound probe to the interior of a subject, receives the reflected waves, and analyzes the reflected waves to display an ultrasound image of the interior of the subject. The subject is a living body of a patient or the like.

The ultrasonic diagnostic apparatus is used not only to display an ultrasound image of a living body inside a subject but also to insert a puncture needle into a target position while visually recognizing the puncture needle and the position of a specific site (target) in the subject. The puncture needle is a hollow needle which is used when a sample of a target in the subject is collected, moisture or the like is discharged, or a drug, a marker, or the like is injected or indwelled in the target. Thus, it is possible to quickly, reliably, and easily perform treatment on the target in the subject.

In recent years, echo-guided puncture manipulations such as nerve block and central vein puncture have attracted attention. In the central vein puncture, in order to avoid complications due to erroneous puncture, it is required to reduce the difficulty of the puncture technique. There is known an ultrasonic diagnostic apparatus that assists a puncture technique for the purpose of reducing difficulty.

For example, an ultrasonic diagnostic apparatus is known in which an ultrasound image and an optical image captured by an optical camera attached to an ultrasound probe are displayed side by side (see Japanese Unexamined Patent Publication No. 2023-121441). In a case where a puncture needle is used, the ultrasonic diagnostic apparatus allows the orientation of the optical image to be inverted vertically and horizontally on the basis of an operation input by a user such as a physician. A user manually changes the orientation of the optical image in accordance with the insertion direction of the puncture needle. Therefore, the user can intuitively recognize the insertion mode of the puncture needle.

### SUMMARY OF THE INVENTION

However, in actual medical care, it is troublesome for a user to adjust the orientation of the optical image by an operation input to a display part or an operation part of the ultrasonic diagnostic apparatus main body. In particular, the user holds an ultrasound probe and a puncture needle and is occupied with both hands. Therefore, an operation input for changing the orientation of the optical image cannot be performed. In addition, no operation input can be performed in the first place when the ultrasonic diagnostic apparatus main body and the user are far from each other. Therefore, there is a demand for easily and appropriately setting the orientation of the optical image.

An object of the present invention is to easily and appropriately set the orientation of a reference image such as an optical image to be displayed together with an ultrasound image of puncture.

To achieve at least one of the abovementioned objects, according to an aspect of the present invention, ultrasonic diagnostic apparatus reflecting one aspect of the present invention is an ultrasonic diagnostic apparatus comprising:
an ultrasound image generator that generates ultrasound image data from reception signals of an ultrasound probe that transmits and receives ultrasound waves to and from a subject;
an optical image capturer that generates optical image data by optically imaging puncture into the subject using a puncture needle; and
a hardware processor that simultaneously displays an ultrasound image of the ultrasound image data and a reference image based on the optical image data, detects a position of a user in an optical image of the optical image data, and sets an orientation of the reference image to be displayed so that the detected position of the user is located on a lower side.

To achieve at least one of the abovementioned objects, according to another aspect of the present invention, image display method reflecting one aspect of the present invention is an image display method comprising:
ultrasound image generating of generating ultrasound image data from reception signals of an ultrasound probe that transmits and receives ultrasound waves to and from a subject;
optical imaging of optically imaging puncture into the subject using a puncture needle to generate optical image data; and
controlling of simultaneously displaying an ultrasound image of the ultrasound image data and a reference image based on the optical image data, detecting a position of a user in an optical image of the optical image data, and setting an orientation of the reference image to be displayed such that the detected position of the user is located on a lower side.

To achieve at least one of the abovementioned objects, according to another aspect of the present invention, recording medium reflecting one aspect of the present invention is a non-transitory recording medium storing a computer-readable program for a computer of an ultrasonic diagnostic apparatus comprising: an ultrasound image generator that generates ultrasound image data from reception signals of an ultrasound probe that transmits and receives ultrasound waves to and from a subject; and an optical image capturer that optically captures puncture into the subject using a puncture needle to generate optical image data, the program causing the computer to perform
controlling of simultaneously displaying an ultrasound image of the ultrasound image data and a reference image based on the optical image data, detecting a position of a user in an optical image of the optical image data, and setting an orientation of the reference image to be displayed such that the detected position of the user is located on a lower side.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a diagram illustrating an external configuration of an ultrasonic diagnostic apparatus of an embodiment according to the present invention;

FIG. 2 is a block diagram illustrating a functional configuration of the ultrasonic diagnostic apparatus;

FIG. 3 is a perspective view illustrating the ultrasound probe, the puncture needle, the optical camera, and the laser pointer of the crossing method of the first embodiment;

FIG. 4 is a perspective view illustrating the ultrasound probe, the puncture needle, the optical camera, and the laser pointer of the parallel method of the first embodiment;

FIG. 5 shows a composite image;

FIG. 6 is a diagram illustrating a table of a schematic diagram illustrating an arrangement relationship of objects in a parallel method, an crossing method, a camera viewpoint, and an optical image orientation for each piece of pattern information;

FIG. 7 is a diagram illustrating an example of an optical image;

FIG. 8 is a diagram illustrating an example of an optical image;

FIG. 9 is a flowchart illustrating the first optical image setting process;

FIG. 10 is a perspective view illustrating the ultrasound probe, the puncture needle, the optical camera, and the laser pointer of the crossing method of the second embodiment;

FIG. 11 is a perspective view illustrating another example of the ultrasound probe, the puncture needle, the optical camera, and the laser pointer of the crossing method of the second embodiment;

FIG. 12 is a flowchart illustrating the second optical image setting process;

FIG. 13 shows a composite image;

FIG. 14 shows a composite image;

FIG. 15 shows a composite image;

FIG. 16 shows a composite image;

FIG. 17 is a diagram illustrating a two dimensional code;

FIG. 18 is a flowchart illustrating third optical image setting process; and

FIG. 19 is a diagram illustrating a user and a sound input section.

### DETAILED DESCRIPTION

Hereinafter, embodiments of the present invention will be described with reference to the drawings. Advantages and features provided by one or more embodiments of the present invention will be more fully understood from the following detailed description and the accompanying drawings. However, these drawings are for illustration purposes only. Therefore, it is not intended to define the limits of the present invention. The scope of the invention is not limited to the disclosed embodiments. Hereinafter, first to fourth embodiments and first and second modification examples of the present invention will be described with reference to the drawings. However, the scope of the present invention is not limited to the disclosed embodiment.

### First Embodiment

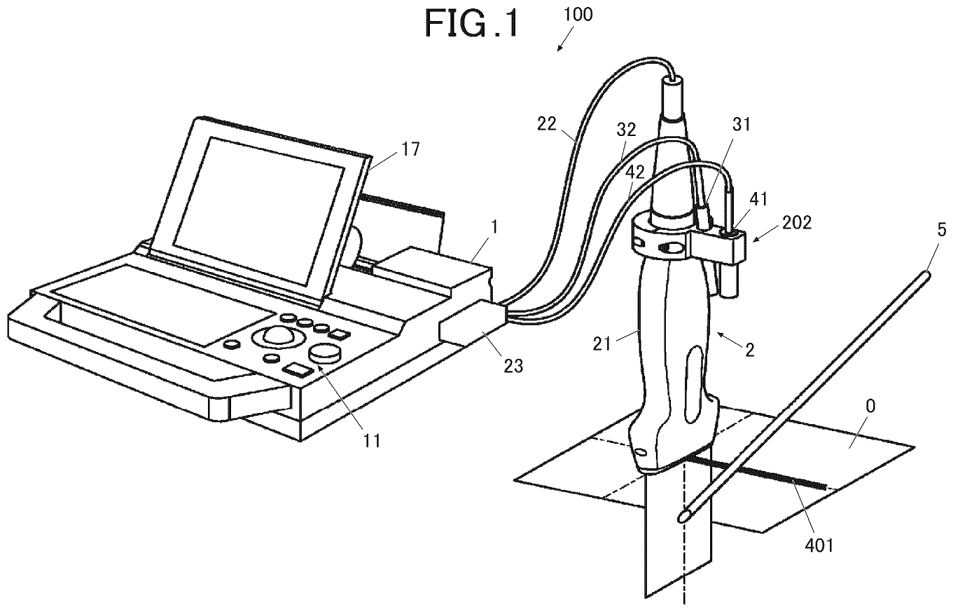

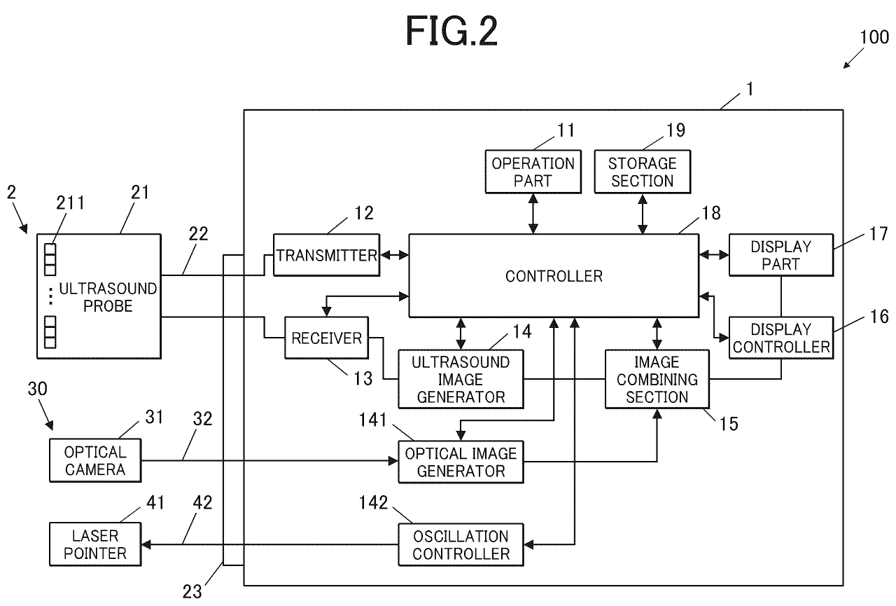

A first embodiment of the present invention will be described with reference to FIGS. 1 to 9. First, a configuration of an apparatus according to the present embodiment will be described with reference to FIGS. 1 and 2. FIG. 1 illustrates the external configuration of an ultrasonic diagnostic apparatus 100 according to the present embodiment. FIG. 2 is a block diagram showing a functional configuration of the ultrasonic diagnostic apparatus 100.

The ultrasonic diagnostic apparatus 100 of the present embodiment is provided in a medical facility such as a hospital. In the present embodiment, the ultrasonic diagnostic apparatus 100 is used for puncture work by a user such as a doctor or a technician using the puncture needle 5. As illustrated in FIG. 1, the ultrasonic diagnostic apparatus 100 includes an ultrasonic diagnostic apparatus main body 1, an ultrasound probe 2, an optical camera 31, and a laser pointer 41. The optical camera 31 and the laser pointer 41 are connected to the ultrasonic diagnostic apparatus main body 1 via the cables 32 and 42, respectively.

The ultrasound probe 2 is connected to the ultrasonic diagnostic apparatus main body 1. The ultrasound probe 2 transmits an ultrasound (transmission ultrasound) to the inside of a subject such as a living body of a patient, and receives a reflected wave (reflected ultrasound: echo) of the ultrasound reflected inside the subject. The ultrasound probe 2 includes an ultrasound probe main body 21, a cable 22, and a connector 23. The ultrasound probe main body 21 is a head part of the ultrasound probe 2, and transmits and receives ultrasound. The cable 22 is connected to the ultrasound probe main body 21 and the connector 23. The cable 22 is a cable through which a drive signal for the ultrasound probe main body 21 and a reception signal of ultrasound flow. The connector 23 is a plug connector for establishing a connection with a receptacle connector (not illustrated) of the ultrasonic diagnostic apparatus main body 1. Here, the connector 23 is a common connector for the cable 32 of the optical camera 31 and the cable 42 of the laser pointer 41.

The ultrasonic diagnostic apparatus main body 1 is connected to the ultrasound probe main body 21 via the connector 23 and the cable 22. The ultrasonic diagnostic apparatus main body 1 transmits a drive signal, which is an electrical signal, to the ultrasound probe main body 21 to direct the ultrasound probe main body 21 to transmit transmission ultrasound waves to the subject. The ultrasound probe 2 generates a reception signal, which is an electrical signal, according to the reflected ultrasound waves from the inside of the subject received by the ultrasound probe main body 21. The ultrasonic diagnostic apparatus main body 1 images the internal state of the subject as ultrasound image data on the basis of the reception signal generated by the ultrasound probe 2.

The ultrasound probe main body 21 includes a transducer 211 (FIG. 2) on a distal end side. The number of transducers 211 can be arbitrarily set, and is actually, for example, one hundred ninety-two. The plurality of transducers are arranged in a one dimensional array in, for example, a scanning direction (an azimuth direction or a long axis direction). Note that the transducer may be arranged in a two dimensional array. In the present embodiment, a linear scanning type electronic scanning probe is adopted as the ultrasound probe 2. However, the ultrasound probe 2 may be of either an electronic scanning type or a mechanical scanning type. In addition, the ultrasound probe 2 may be of any of a linear scanning type, a sector scanning type, and a convex scanning type. Furthermore, the ultrasonic diagnostic apparatus main body 1 and the ultrasound probe 2 may be configured to perform wireless communication instead of wired communication via the cable 22. The wireless communication is an ultra-wide band (UWB), for example.

The ultrasonic diagnostic apparatus main body 1 includes an operation part 11 and a display part 17. The operation part 11 accepts various operation inputs from the user. The operation part 11 includes operation elements such as a push button, an encoder, a lever switch, a joystick, a trackball, a keyboard, a touch pad, and a multifunction switch.

The display part 17 includes a display panel such as a liquid crystal display (LCD) and an electro-luminescence (EL) display. The display part 17 displays display information such as an ultrasound image based on the ultrasound image data. In particular, the display part 17 displays a composite image of an ultrasound image by the ultrasound probe 2 and an optical image by the optical camera 31.

In the ultrasonic diagnostic apparatus 100, the optical camera 31 and the laser pointer 41 have a predetermined positional relationship with the ultrasound probe 2 (ultrasound probe main body 21). In this manner, the optical camera 31 and the laser pointer 41 are attached to the ultrasound probe main body 21 via the detachable attachment 202. The positional relationship of the optical camera 31 and the laser pointer 41 with respect to the ultrasound probe 2 are determined by the attachment 202. Provided that the attachment 202 may enable adjustment of the posture of the optical camera 31 and the laser pointer 41.

The attachment 202 is, for example, a screw-fixing type pinching member, and is attached to the ultrasound probe main body 21 so as to pinch the ultrasound probe main body 21 from both right and left sides. The attachment 202 is made of, for example, a material that can withstand a disinfectant, for example, POM (polyacetal). Furthermore, when the attachment 202 is attached to the ultrasound probe main body 21, the ultrasound probe 2, the optical camera 31, and the laser pointer 41 are in a state of being aligned. Therefore, a probe notch (not shown) is provided on the outer surface of the ultrasound probe main body 21. A projection (not shown) to be fitted into the probe notch is provided on the inner peripheral surface of the attachment 202.

The optical camera 31 is, for example, a general fiberscope camera that acquires an optical image signal by a built-in imaging element. The optical camera 31 includes, for example, a zoom magnification lens and can magnify and image an imaging target (here, a body surface region of the subject 0). The optical camera 31 is attached to the proximal end side of the ultrasound probe main body 21.

The laser pointer 41 is, for example, a general laser diode that outputs visible-color laser light (e.g., red laser light having wavelength 635 nm to 690 nm). The laser pointer 41 is attached to a proximal end side of the ultrasound probe main body 21. The laser pointer 41 emits laser light onto the body surface of the subject 0 to form a predetermined projection image 401. The laser pointer 41 outputs laser light so that an irradiation shape of a projection image 401 that is laser light on a body surface in the subject 0 becomes a line shape by a built-in diffraction grating or a slit.

In the puncture work, for example, it is assumed that the puncture needle 5 is inserted into the subject 0 by a user in a free-hand manner. A user brings a transmission/reception surface of an ultrasound beam of the ultrasound probe 2 into contact with a body surface of the subject 0 and operates the ultrasonic diagnostic apparatus 100, to obtain ultrasound image data inside the subject 0. The user looks at the display part 17 and checks the position of the puncture target, such as a blood vessel, a tissue, or a lesion, in the subject 0 appearing in the ultrasound image in the composite image. The user grasps the target insertion position and the target posture of the puncture needle 5 when the puncture needle 5 is inserted into the subject 0 from the optical image of the composite image, and performs the puncture work. At this time, a projection image 401 is formed on the body surface of the subject by the laser light of the laser pointer 41 in the optical image. The projection image 401 shows the target insertion position and the target posture of the puncture needle 5. Thus, the user can perform accurate puncture work.

As shown in FIG. 2, the ultrasonic diagnostic apparatus main body 1 includes an operation part 11, a transmitter 12, a receiver 13, an ultrasound image generator 14, an optical image generator 141, an oscillation controller 142, an image combining section 15, a display controller 16, a display part 17, a controller 18 (hardware processor), and a storage section 19. The optical camera 31 and the optical image generator 141 function as the optical image capturer 30.

The operation part 11 receives various operation inputs from the user and outputs the operation signals to the controller 18. The operation part 11 may include a touch screen integrally formed on the display screen of the display part 17 so as to receive a user's touch input.

The transmitter 12 supplies a drive signal, which is an electrical signal, to the ultrasound probe 2 in accordance with the control of the controller 18 to cause the ultrasound probe 2 to generate a transmission ultrasound wave. The transmitter 12 drives, for example, a consecutive part (e.g., sixty-four) of a plurality of (e.g., one hundred ninety-two) transducers arrayed in the ultrasound probe 2 to generate transmission ultrasound waves. Then, the transmitter 12 performs scanning by shifting the transducer to be driven in the scanning direction every time the transmission ultrasound is generated.

The receiver 13 receives a reception signal which is an analog electrical signal received from the ultrasound probe 2, amplifies the reception signal, and performs analog-to-digital (AD) conversion on the reception signal under the control of the controller 18. The receiver 13 provides a delay time to the digital reception signal after the AD conversion for each individual path corresponding to each transducer to adjust the time phase, and performs addition (delay-and-sum) to generate sound ray data.

Under the control of the controller 18, the ultrasound image generator 14 performs envelope detection processing, logarithmic compression, and the like on the sound ray data from the receiver 13. The ultrasound image generator 14 adjusts the dynamic range and the gain of the sound ray data after processing such as logarithmic compression, converts the data into brightness, and generates image data (B) mode image data. That is, the B-mode image data is tomographic image data in which the intensity of a reception signal in a case where the image mode is the B mode is represented by luminance. Provided that the ultrasound image generator 14 may be configured to generate color Doppler image data or the like and superimpose it on the B-mode image data.

Under the control of the controller 18, the optical image generator 141 acquires an optical image signal from the optical camera 31 via the cable 32 and generates optical image data. The optical image generator 141 continuously generates optical image data in sections of frames, for example, on the basis of optical image signals sequentially obtained from the optical camera 31, to generate optical image data of a moving image. Note that the optical image generator 141 may be built in the optical camera 31.

The oscillation controller 142 controls, under the control of the controller 18, a driving current flowing through the laser diode of the laser pointer 41 to control turning on/off of the output operation of the laser light.

Under the control of the controller 18, the image combining section 15 acquires the ultrasound image data from the ultrasound image generator 14 and acquires the optical image data from the optical image generator 141. The image combining section 15 generates composite image data for displaying the ultrasound image of the ultrasound image data and the optical image of the optical image data in the same display screen. The image combining section 15 outputs the generated composite image data to the display controller 16. The image combining section 15 generates composite image data in real time each time new ultrasound image data is acquired and/or each time new optical image data is acquired. The image combining section 15 outputs the generated composite image data to the display controller 16.

Note that the image combining section 15 may be capable of changing the display mode of the ultrasound image and/or the optical image in the composite image in accordance with user setting contents input to the controller 18 or the operation part 11. Further, the image combining section 15 may generate the composite image data after performing predetermined image processing on the input ultrasound image data or the input optical image data.

The display controller 16 is, for example, a digital scan converter (DSC). Under the control of the controller 18, the display controller 16 performs processing such as coordinate conversion on the composite image data received from the image combining section 15 to convert the composite image data into an image signal for display.

Under the control of the controller 18, the display part 17 displays the composite image on the display panel in accordance with the image signal output from the display controller 16. Furthermore, the display part 17 displays various kinds of display information input from the controller 18 on the display panel.

The controller 18 includes, for example, a central processing unit (CPU), a read only memory (ROM), and a random access memory (RAM). The controller 18 reads various processing programs stored in the ROM, loads the programs to the RAM, and controls the components of the ultrasonic diagnostic apparatus 100 in accordance with the loaded programs and the CPU. The ROM includes a nonvolatile memory such as a semiconductor. The ROM stores a system program corresponding to the ultrasonic diagnostic apparatus 100, various processing programs executable on the system program, various data such as a gamma table, and the like. In particular, the ROM stores a first optical image setting program for executing first optical image setting process described later. These programs are stored in the RAM in the form of computer-readable program codes. The CPU sequentially executes operations according to the program code on the RAM. The RAM forms a work area in which various programs executed by the CPU and data related to these programs are temporarily stored.

The storage section 19 is a storage section such as a hard disk drive (HDD), a solid state drive (SSD) or the like that stores information such as ultrasound image data in a writable and readable manner.

With respect to each section included in the ultrasonic diagnostic apparatus 100, some or all of the functions of each functional block can be implemented as a hardware circuit such as an integrated circuit. The integrated circuit is, for example, a large scale integration (LSI). The LSI may be referred to as an integrated circuit (IC), a system LSI, a super LSI, or an ultra LSI, depending on the degree of integration. Further, the method of circuit integration is not limited to LSI. The integrated circuit method may be implemented by a dedicated circuit or a processor. As a method of circuit integration, a field programmable gate array (FPGA) or a reconfigurable processor in which connections and settings of circuit cells in an LSI can be reconfigured may be used. Furthermore, some or all of the functions of the functional blocks may be implemented by software. In this case, the software is stored in one or each of a storage medium such as a ROM, an optical disc, a hard disk, or the like, and the software is executed by the arithmetic processor.

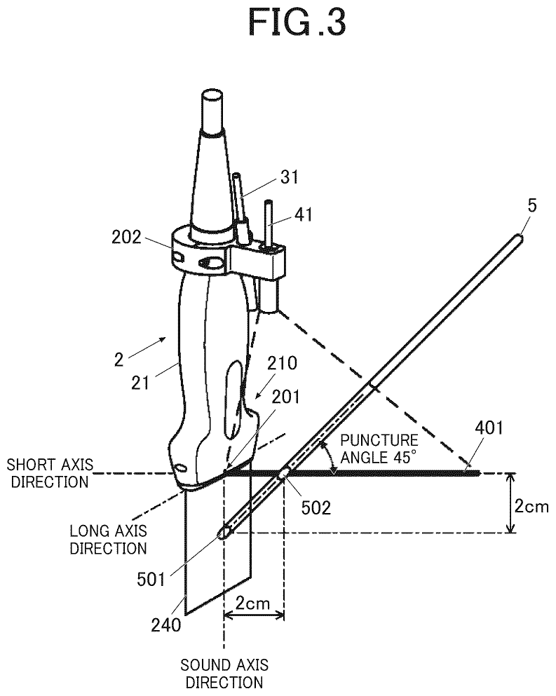

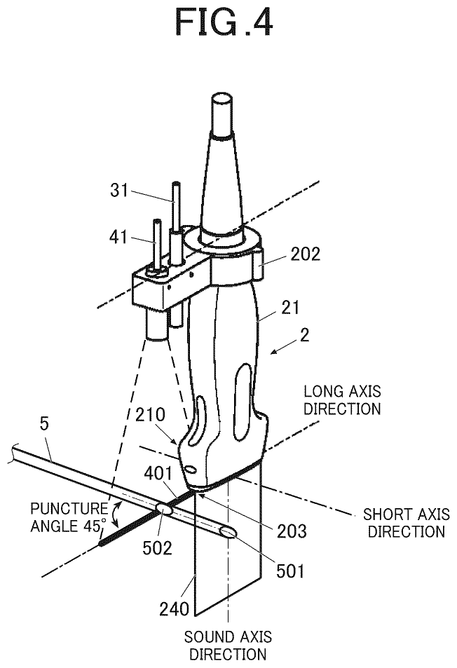

Next, puncturing by the crossing method and the parallel method as puncturing modes will be described with reference to FIG. 3 and FIG. 4. FIG. 3 is a perspective view illustrating the ultrasound probe 2, the puncture needle 5, the optical camera 31, and the laser pointer 41 of the crossing method of the present embodiment. FIG. 4 is a perspective view illustrating the ultrasound probe 2, the puncture needle 5, the optical camera 31, and the laser pointer 41 of the parallel method of the present embodiment.

The crossing method is a method of puncturing with the long axis direction of the ultrasound probe 2 and the puncture needle 5 orthogonal to each other. The long axis direction of the ultrasound probe 2 is the one dimensional arrangement direction of the transducer 211, and is the scanning direction (azimuth direction). The short axis direction of the ultrasound probe 2 is a direction orthogonal to the long axis direction and is an elevation direction. The parallel method is a method of puncturing with the long axis direction of ultrasound probe 2 and puncture needle 5 being parallel to each other.

The puncture by the crossing method will be described with reference to FIG. 3. FIG. 3 is a perspective view of the ultrasound probe 2 as viewed obliquely from above. The optical camera 31 and the laser pointer 41 are attached side by side in the short axis direction correspondingly to a central position 201 in the long axis direction of the ultrasound probe main body 21 by an attachment 202. The optical camera 31 is attached so that the tip part 210 of the ultrasound probe main body 21, the projection image 401, and the observation target site appear in the optical image. The observation target site is an observation target site of an ultrasound image of the body surface of the subject 0. However, the short axis direction in FIG. 3 is shifted in the drawing for easy viewing.

The projection image 401 guides a target position and a target posture of the puncture needle 5 with the tip end portion 210 as a reference position in the optical image. For example, the projection image 401 has a line shape extending in the short axis direction from the center position 201 as a start point on the body surface of the subject 0. The target posture of the puncture needle 5 is, for example, an appropriate orientation of the puncture needle 5 in a plan view (meaning a field of view from above the body surface of the subject 0).

The puncture needle 5 is inserted into, for example, a target site 501 in the subject 0 via the target insertion position 502. Here, the extending direction of the projection image 401 of the line-shaped laser light is a target posture of the puncture needle 5 when the puncture needle 5 is inserted into the subject 0. For example, a position separated from the target site 501 by 2 cm in the vertical direction and by 2 cm in the short axis direction is the target insertion position 502. At this time, the elevation angle (puncture angle) of the puncture needle 5 with respect to the short axis direction is 45°. However, the puncture angle, the target site 501, and the target insertion position 502 are not limited to these examples.

The puncture by the parallel method will be described with reference to FIG. 4. FIG. 4 is a perspective view of the ultrasound probe 2 as viewed obliquely from above. The optical camera 31 and the laser pointer 41 are attached side by side on the long axis direction correspondingly to a center position 203 in the short axis direction of the ultrasound probe main body 21 by an attachment 202. The optical camera 31 is attached so that the distal end portion of the ultrasound probe main body 21, the projection image 401, and the observation target site are reflected in the optical image. The ultrasound image obtained by the ultrasound probe 2 is an ultrasound image corresponding to the scan section 240 of the ultrasound probe 2. The scan section 240 is a section passing through the sound axis direction of the ultrasound orthogonal to the long axis direction and the short axis direction.

The projection image 401 guides a target position and a target posture of the puncture needle 5 with the tip end portion 210 as a reference position in the optical image. For example, the projection image 401 has a line shape extending in the long axis direction from the central position 201 as a starting point on the body surface of the subject 0.

The puncture needle 5 is inserted into, for example, a target site 501 in the subject 0 via the target insertion position 502. Here, the extending direction of the projection image 401 of the line-shaped laser light is a target posture of the puncture needle 5 when the puncture needle 5 is inserted into the subject 0. For example, a position separated from the target site 501 by 2 cm in the vertical direction and by 2 cm in the long axis direction is the target insertion position 502. At this time, the elevation angle (puncture angle) of the puncture needle 5 with respect to the short axis is 45°. However, the puncture angle, the target site 501, and the target insertion position 502 are not limited to these examples.

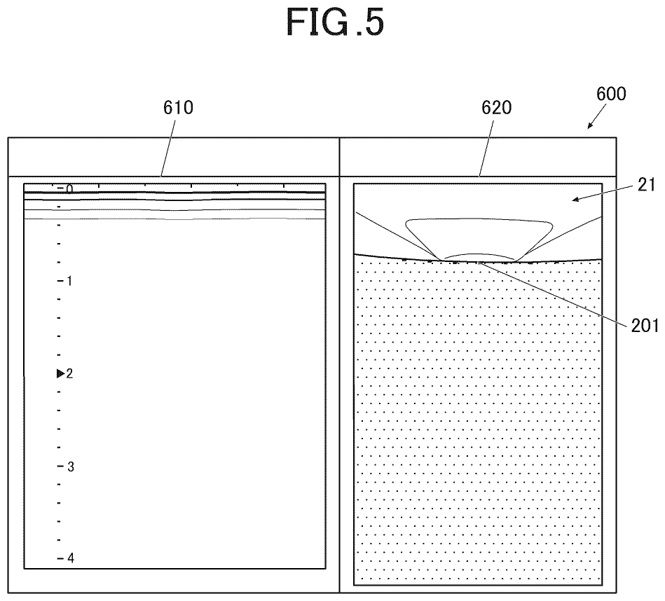

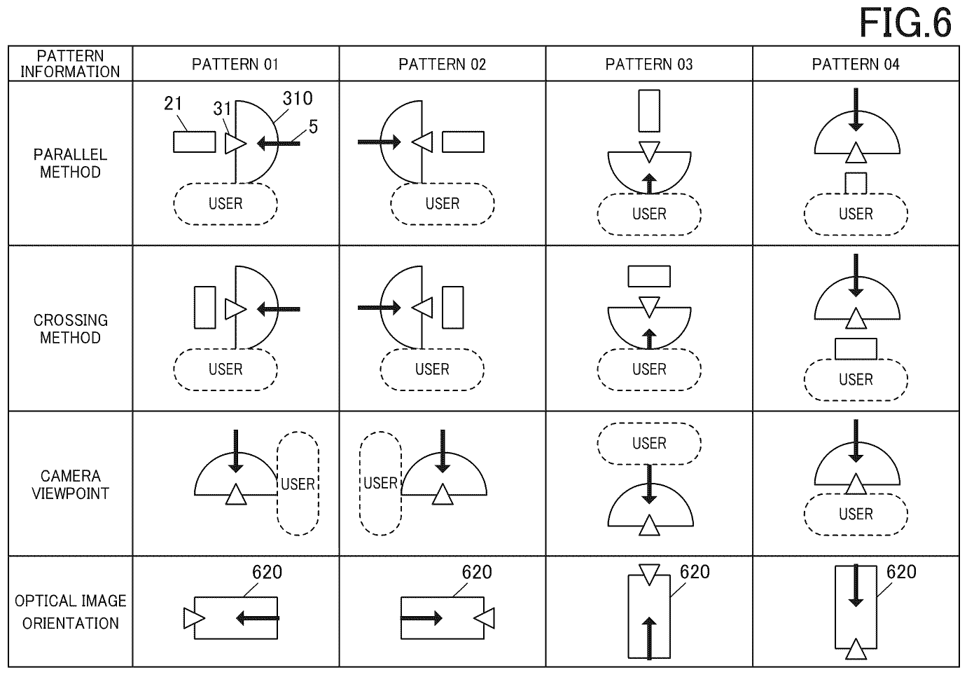

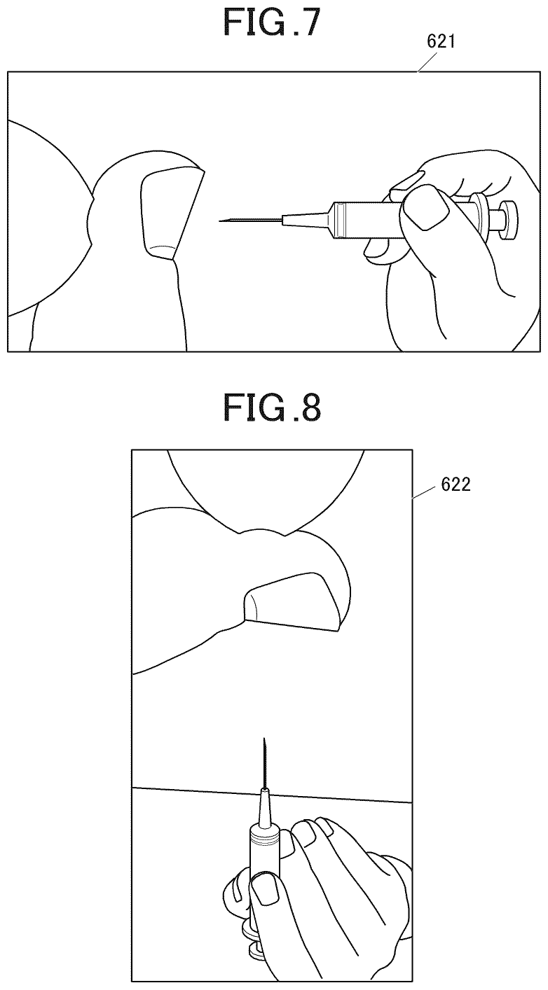

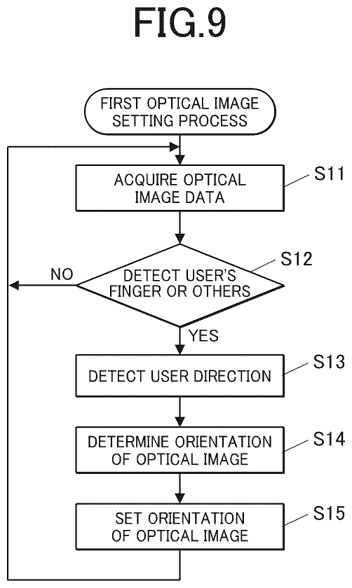

Next, with reference to FIGS. 5 to 9, operation of the ultrasonic diagnostic apparatus 100 will be described. FIG. 5 is a diagram illustrating a composite image 600. FIG. 6 is a diagram illustrating a table of a schematic diagram illustrating an arrangement relationship of objects in a parallel method, an crossing method, a camera viewpoint, and an optical image orientation for each piece of pattern information. FIG. 7 is a view illustrating an example of an optical image. FIG. 8 is a view illustrating an example of an optical image. FIG. 9 is a flowchart illustrating first optical image setting process.

A composite image 600 as an example of a composite image of composite image data generated by the image combining section 15 will be described with reference to FIG. 5.

The composite image 600 includes an ultrasound image 610 and an optical image 620. The ultrasound image 610 is a B-mode image of the subject 0 based on the ultrasound image data generated by the ultrasound image generator 14. The optical image 620 is an optical image based on the optical image data generated by the optical image generator 141. The optical image 620 is, for example, a captured image in a state where a user inserts a puncture needle upward from below by a crossing method. The optical image 620 includes a central position 201 of the ultrasound probe main body 21 and the body surface of the subject 0. The projection image 401 is omitted from the optical image 620. Furthermore, the optical image 620 may have a first center line for indicating the center of the optical image in the horizontal direction and a second center line for indicating the center of the optical image in the vertical direction. Although the user is not reflected in the optical image 620, the user is located in the lower direction, which is appropriate direction for puncture.

Hereinafter, up, down, left, and right directions are defined as up, down, left, and right directions of an image plane or directions of an object such as a user corresponding to the up, down, left, and right directions.

The composite image 600 has a configuration in which the ultrasound image 610 and the optical image 620 are divided into left and right on the display screen, but is not limited thereto. For example, in the composite image, the ultrasound image and the optical image may be divided into upper and lower parts on the display screen, or the positions of the ultrasound image and the optical image may be switched.

Next, an appropriate orientation of an optical image to be displayed will be described with reference to FIG. 6. In FIG. 6, the vertical axis represents the items of the parallel method, the crossing method, and the camera viewpoint, and the arrangement relationship of the objects related to the optical imaging in each item is shown in a schematic diagram. The objects are the user, the ultrasound probe main body 21, the optical camera 31, and the puncture needle 5. The schematic diagram is a diagram viewed from a vertical direction of a plane of a body surface of the subject 0. Here, the laser pointer 41 and its projected image 401 are omitted.

Each schematic diagram of FIG. 6 is a plan view. In FIG. 6, the ultrasound probe main body 21 is indicated by a rectangle. The longitudinal direction of the rectangle corresponds to the long axis direction of the ultrasound probe main body 21. Similarly, the short direction of the rectangle corresponds to the short axis direction of the ultrasound probe main body 21. The optical camera 31 is indicated by an isosceles triangle. An apex angle of the isosceles triangle is defined as an imaging direction (optical axis direction) of the optical camera 31. Further, an imaging range 310 of the optical camera 31 is indicated by a semicircle. The approximate center of the semicircle is defined as the imaging surface of the optical camera 31. The puncture needle 5 is indicated by an arrow. The direction of the arrow is defined as the insertion direction of the puncture needle 5.

In FIG. 6, the schematic diagram of the parallel method is a diagram in which the user is arranged on the lower side. The parallel method is divided into four states according to the position of the user with respect to the ultrasound probe main body 21, the optical camera 31, and the puncture needle 5. Similarly, in the schematic diagram of the crossing method, the user is arranged on the lower side. The crossing method is also divided into four states according to the position of the user with respect to the ultrasound probe main body 21, the optical camera 31, and the puncture needle 5.

In FIG. 6, the schematic diagram of the camera viewpoint is a schematic diagram in which the optical camera 31 is located on the lower side and the upward direction is the imaging direction. In the schematic diagram of the camera viewpoint, the ultrasound probe main body 21 is omitted. Each schematic diagram of the parallel method and the crossing method is classified into four pieces of pattern information according to the schematic diagram of the camera viewpoint. Four pieces of pattern information are referred to as patterns 01, 02, 03, and 04.

In FIG. 6, the schematic diagram of the optical image orientation is a view showing the arrangement of the optical image and the object corresponding to the schematic diagram of the camera viewpoint. In the schematic diagram of the optical image orientation, the optical camera 31 and the puncture needle 5 are illustrated. The optical image 620 is indicated by a large rectangle. Further, in the schematic diagram of the optical image orientation, the user is arranged on the lower side. The optical image 620 is thus oriented in an appropriate display direction of the user viewpoint. The optical image 620 in an appropriate direction is determined by the user and the direction (position) of the puncture needle 5 with respect to the optical image 620.

FIGS. 7 and 8 show an example of the optical image 620. An optical image 620 of FIG. 7 is an optical image corresponding to the pattern 01 of the parallel method of FIG. 6. In the optical image 620 of FIG. 7, the ultrasound probe main body 21, the hand of the user, the puncture needle 5 (the injector including the puncture needle 5), and the body surface of the subject 0 are shown. An optical image 620 in FIG. 7 is an optical image in an appropriate direction of a user viewpoint from the lower side. However, if the displayed optical image 620 of FIG. 7 is in a rotated orientation in plan view, it will not be an appropriate image for the user viewpoint. Therefore, it is necessary to change the orientation of the displayed optical image 620 having a different orientation to the orientation of the optical image 620 of FIG. 7.

The optical image 620 of FIG. 8 is an optical image corresponding to the pattern 03 of the parallel method of FIG. 6. The optical image 620 in FIG. 8 also shows the ultrasound probe main body 21, the user's hand, (the injector having) the puncture needle 5, and the body surface of the subject 0. The displayed optical image 620 of FIG. 8 is an optical image of the proper direction of the user's viewpoint from below. However, the optical image 620 of FIG. 8 is also not an appropriate image of the user viewpoint in a case where the optical image 620 is in a rotated orientation in a plan view. Therefore, it is necessary to change the orientation of the displayed optical image 620 having a different orientation to the orientation of the optical image 620 in FIG. 8.

Next, the first optical image setting process executed by the ultrasonic diagnostic apparatus 100 will be described with reference to FIG. 9. The optical camera 31 and the laser pointer 41 are attached to the ultrasound probe 2 in advance. In an ultrasonic diagnostic apparatus 100, the controller 18 executes composite image display processing. As the composite image display processing, the controller 18 scans the subject by the ultrasound probe 2, generates a projection image by the laser pointer 41, and images the imaging range by the optical camera 31. The controller 18 generates ultrasound image data with the ultrasound image generator 14 and generates optical image data with the optical image generator 141. The controller 18 generates composite image data by the image combining section 15 and displays a live composite image based on the composite image data on the display part 17. The user performs ultrasound-guided puncture. Specifically, the user inserts the puncture needle 5 into the target site of the subject by the parallel method or the crossing method while visually checking the ultrasound image and the optical image of the composite image.

The first optical image setting process is a process of setting the orientation of the composite image of the optical image data generated in the combined image process described above to an appropriate direction on the basis of the optical image. For example, triggered by the execution of the composite image display processing, the controller 18 executes the first optical image setting process in accordance with the first optical image setting program in the ROM.

First, the controller 18 acquires the generated optical image data from the optical image generator 141 (step S11). The controller 18 performs image analysis on the optical image data acquired in step S11 and determines whether or not the user's fingers, hands, wrists, or arms at the end of the puncture needle 5 are detected (step S12). In a case where the user's fingers or the like (fingers, hands, wrists, arms) are not detected (step S12; NO), the process proceeds to step S11. When the user's finger or the like is detected (step S12; YES), the controller 18 detects, from the detected user's finger or the like, the direction (position) of (the body of) the user relative to the optical image (step S13).

If the optical image has a sufficient angle of view in step S12, the controller 18 may analyze the optical image data to detect the user's body and face. With this structure, in step S13, the controller 18 detects the direction of the user relative to the optical image from the recognized user's body or the like.

The controller 18 determines the appropriate optical image orientation of the user viewpoint relative to the current optical image based on the user direction detected in step S13 (step S14). That is, it is determined that the direction of the detected user of the optical image to be displayed is set to the lower side. The controller 18 sets the orientation of the optical image of optical image data to the direction determined in step S14 (step S15). Thereafter, the processing proceeds to step S11. In the setting of the step S15, for example, the image combining section 15 is set to combine the optical image of the optical image data in the determined direction. However, it is not limited thereto, and for example, a configuration may be adopted in which the optical image data itself is changed so that the optical image is set in the determined direction.

In addition, since the process proceeds to step S11 after step S15, when the position of the user changes, the orientation of the optical image also changes (follows) in real time.

As illustrated in the first optical image setting process, the optical image data does not need to be acquired at once in step S11. The user moves (e.g., moves the position or inclines the angle) the ultrasound probe 2 to widen the imaging range of the optical camera 31. Thus, the direction of the user and the direction of the puncture needle 5 may be detected.

As described above, according to the present embodiment, the ultrasonic diagnostic apparatus 100 includes the ultrasound image generator 14, the optical image capturer 30, and the controller 18. The ultrasound image generator 14 generates ultrasound image data from reception signals of the ultrasound probe 2 that transmits and receives ultrasound waves to and from the subject. The optical image capturer 30 optically images the puncture of the puncture needle 5 into the subject and generates optical image data. The controller 18 causes the display part 17 to simultaneously display an ultrasound image of the ultrasound image data and an optical image as a reference image based on the optical image data. The controller 18 also detects the position of the user in the optical image of the optical image data, and sets the orientation of the optical image to be displayed so that the detected position of the user is located on the lower side.

Therefore, it is possible to easily and appropriately set the orientation of the optical image to be displayed together with the ultrasound image of puncture such that the position of the user is on the lower side without time and effort of the operation input of the ultrasonic diagnostic apparatus 100 by the user. Thus, the traveling direction (orientation) of the needle image can be automatically adjusted in accordance with the insertion direction of the puncture needle 5. Therefore, the user can intuitively and easily perform a puncture procedure, and the load on the user can be reduced.

In addition, the controller 18 analyzes the optical image data to determine the position of the user in the optical image. Therefore, it is possible to easily, appropriately, and automatically set, without a user's operation, the orientation of the optical image to be displayed together with the ultrasound image of the puncture such that the position of the user is on the lower side.

### Second Embodiment

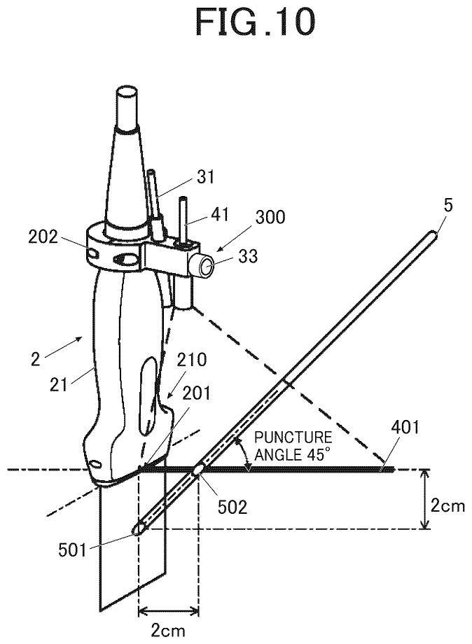

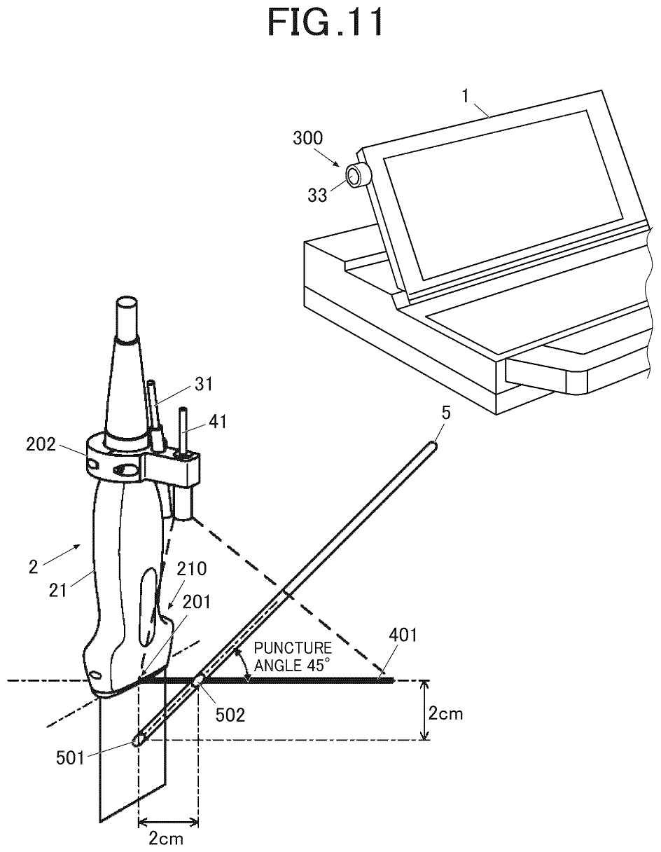

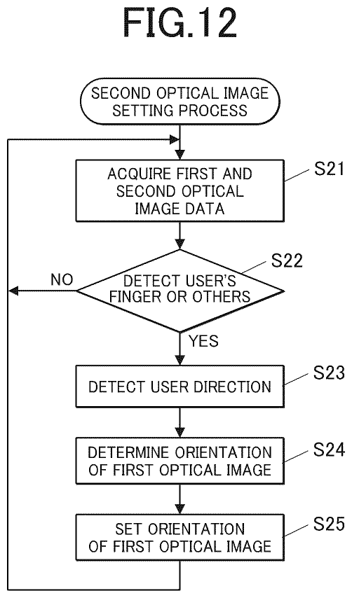

A second embodiment of the present invention will be described with reference to FIGS. 10 to 12. FIG. 10 is a perspective view illustrating the ultrasound probe 2, the puncture needle 5, the optical cameras 31 and 33, and the laser pointer 41 of the crossing method of the present embodiment. FIG. 11 is a perspective view illustrating another example of the ultrasound probe 2, the puncture needle 5, the optical cameras 31 and 33, and the laser pointer 41 of the crossing method in the present embodiment. FIG. 12 is a flowchart illustrating the second optical image setting process.

In the first embodiment described above, the orientation of the optical image to be displayed is set to an appropriate direction using only the optical image data of the optical camera 31. In the present embodiment, two pieces of optical image data from the optical cameras 31 and 33 are used to set the orientation of an optical image to be displayed in an appropriate direction.

In the present embodiment, similarly to the first embodiment, the ultrasonic diagnostic apparatus 100 is used as an apparatus configuration. However, in the ultrasonic diagnostic apparatus 100 of the present embodiment, the description of the configuration of the same parts as those of the ultrasonic diagnostic apparatus 100 of the first embodiment will be omitted, and different parts will be mainly described.

As shown in FIG. 10, the ultrasonic diagnostic apparatus 100 of the present embodiment further includes an optical camera 33. The optical image capturer 300 includes an optical camera 33 and an optical image generator 141. The optical camera 33 is a camera similar to the optical camera 31, and has an imaging direction different from that of the optical camera 31. The optical camera 33 is attached to, for example, the attachment 202. It is preferable that the optical camera 33 includes, for example, a fish-eye lens so as to widen an imaging range. When the imaging range is wide, a user's face, body, and the like can be imaged in addition to the puncture needle 5, the user's hand, and the like. The optical camera 33 is connected to the optical image generator 141 via a cable (not illustrated) and the connector 23.

Under the control of the controller 18, the optical image generator 141 acquires an optical image signal from the optical camera 31 via the cable 32 and generates first optical image data. Similarly, the optical image generator 141 acquires an optical image signal from the optical camera 33 and generates second optical image data.

Under the control of the controller 18, the image combining section 15 acquires the ultrasound image data from the ultrasound image generator 14 and acquires the first and second optical image data from the optical image generator 141. The image combining section 15 generates composite image data of a composite image of the ultrasound image of the ultrasound image data and the first optical image of the first optical image data.

Note that as illustrated in FIG. 11, the optical camera 33 may be configured to be attached to a position other than the attachment 202, such as the ultrasonic diagnostic apparatus main body 1.

In the ROM of the controller 18, a second optical image setting program for executing a second optical image setting process described later is stored instead of the first optical image setting program.

Next, with reference to FIG. 12, the second optical image setting process executed by the ultrasonic diagnostic apparatus 100 of the present embodiment will be described. In advance, as in the first embodiment, the controller 18 executes the composite image display processing. However, in the composite image display processing according to the present embodiment, composite image data of the ultrasound image and the first optical image based on the first optical image data from the optical image generator 141 is displayed on the display part 17. The user inserts the puncture needle 5 into the target site of the subject by the parallel method or the crossing method while visually checking the ultrasound image and the optical image of the composite image.

The second optical image setting process is processing of setting the orientation of the composite image of the generated optical image data in the composite image processing to an appropriate direction based on the optical image. For example, triggered by the execution of the composite image display processing, the controller 18 executes the second optical image setting process in accordance with the second optical image setting program in the ROM.

First, the controller 18 acquires the generated first and second optical image datasets from the optical image generator 141 (step S21). The controller 18 analyzes at least one of the first and second optical image data acquired in step S21 (step S22). In step S22, the controller 18 determines, from the image analysis result, whether or not user's fingers, hands, wrists, and arms at the end of the puncture needle 5 are detected. When the user's finger or the like is not detected (step S22; NO), the process proceeds to step S21. When the user's finger or the like is detected (step S22; YES), the controller 18 detects, from the detected user's finger or the like, the direction (position) of (the body of) the user relative to the first optical image (step S23).

Based on the direction of the user detected in step S23, the controller 18 determines an appropriate orientation of the first optical image from the user's viewpoint with respect to the current first optical image (step S24). The controller 18 sets the orientation of the optical image of the optical image data to the direction determined in step S24 (step S25). Thereafter, the processing proceeds to step S21.

As described above, according to the present embodiment, the optical image capturer 30 as the first optical image capturer and the optical image capturer 300 as the second optical image capturer are provided. An optical image capturer 30 generates first optical image data by optically imaging puncture of a puncture needle into a subject. The optical image capturer 300 generates second optical image data by optically imaging the puncture of the puncture needle into the subject in an imaging direction different from that of the optical image capturer 30. The controller 18 simultaneously displays an ultrasound image of the ultrasound image data and a first optical image as a reference image based on the first optical image data. The controller 18 also performs image analysis on at least one of the first optical image data and the second optical image data to detect the position of the user in the first optical image of the first optical image data. Therefore, it is possible to widen a recognizable imaging range by using the plurality of optical image capturers, and it is possible to more accurately detect the position (direction) of the user in the first optical image.

### Third Embodiment

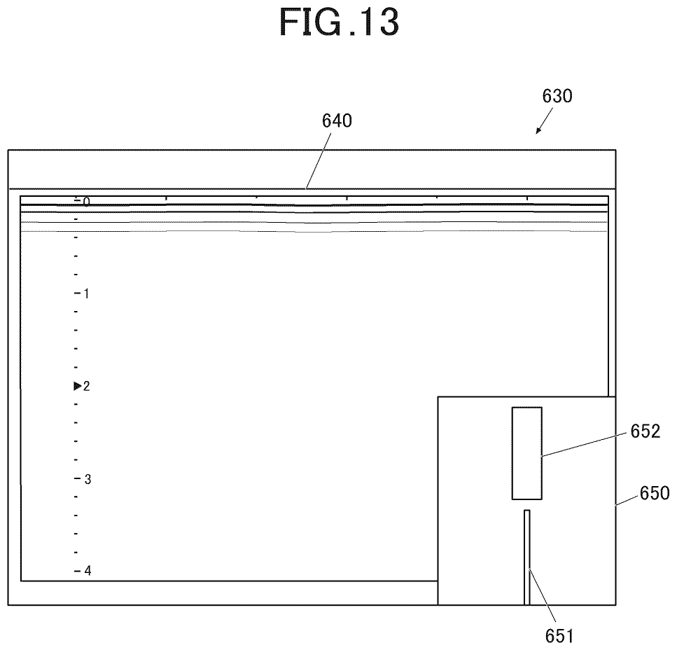

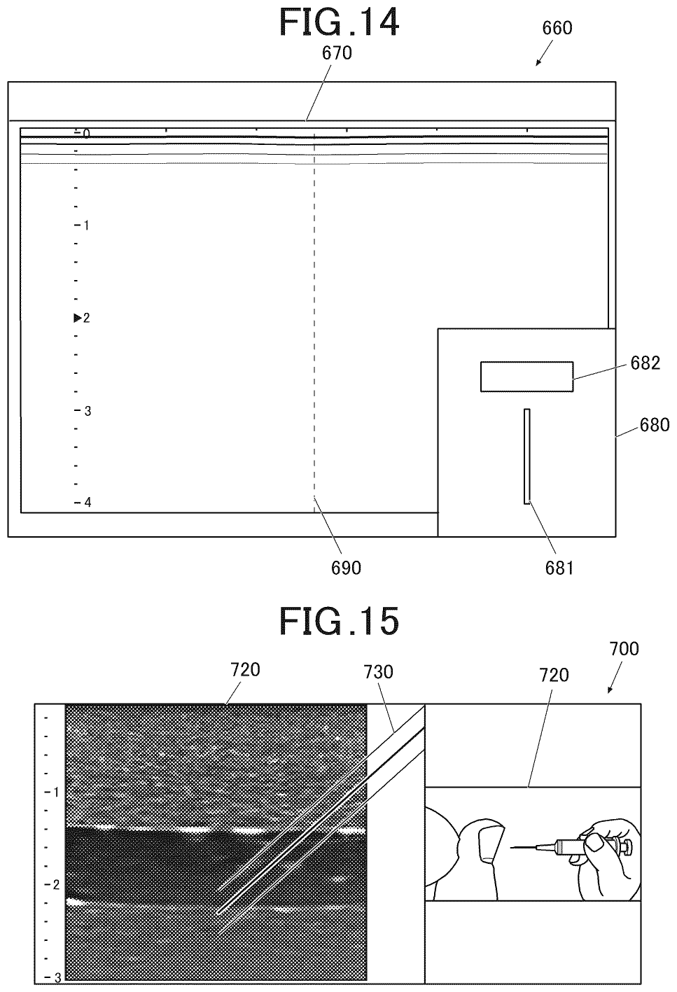

A third embodiment of the present invention will be described with reference to FIGS. 13 to 16. FIG. 13 is a diagram illustrating a composite image 630. FIG. 14 is a diagram illustrating a composite image 660. FIG. 15 is a view illustrating a composite image 700. FIG. 16 is a diagram illustrating a composite image 740.

In the first embodiment, the composite image 600 of the composite image data includes the ultrasound image 610 and the optical image 620. In the present embodiment, the composite image of the composite image data includes an ultrasound image and an illustration image.

In the present embodiment, similarly to the first embodiment, the ultrasonic diagnostic apparatus 100 is used as an apparatus configuration. Provided that an illustration image data generating process is executed instead of the first optical image setting process described above.

An example of a composite image displayed in the present embodiment will be described with reference to FIGS. 13 and 14. As shown in FIG. 13, the composite image 630 is a composite image corresponding to the parallel method, and includes an ultrasound image 640 and an illustration image 650. The illustration image 650 is an image in which only parts of the ultrasound probe main body 21 and the puncture needle 5 are displayed among the schematic diagrams of the parallel method in FIG. 6. Here, it is assumed that the illustration image 650 is an illustration image corresponding to the schematic diagram of the pattern 03 of the parallel method in FIG. 6. The illustration image 650 includes, for example, a needle image 651 of the puncture needle 5 and a probe image 652 of the ultrasound probe main body 21.

As shown in FIG. 14, the composite image 660 is a composite image corresponding to the crossing method, and includes an ultrasound image 670 and an illustration image 680. The illustration image 680 is an image in which only parts of the ultrasound probe main body 21 and the puncture needle 5 are displayed among the schematic diagrams of the crossing method in FIG. 6. Here, it is assumed that the illustration image 680 is an illustration image corresponding to the schematic diagram of the pattern 03 of the crossing method in FIG. 6. The illustration image 680 includes, for example, a needle image 681 of the puncture needle 5 and a probe image 682 of the ultrasound probe main body 21.

Since the illustration image 650 is easier to see than the optical image, the size of the illustration image 650 is smaller than that of the optical image 620 of the composite image 600 in FIG. 5. Therefore, the ultrasound image 640 is larger than the ultrasound image 610 of the composite image 600 and has a large visible range. The same applies to the illustration image 680 and the ultrasound image 670.

Furthermore, the composite image 660 includes a vertical line 690 at the center in the left-right direction as a user interface (UI) for puncture assistance. In the crossing method, the presence of the line 690 makes it easy for the user to confirm the puncture needle portion near the line 690 in the ultrasound image 670. In the parallel method, the needle image in the ultrasound image appears in right and left oblique directions. Therefore, line display is not necessary in the ultrasound image 640.

Next, the illustration image data generating process executed by the ultrasonic diagnostic apparatus 100 will be described with reference to FIG. 9. In the ultrasonic diagnostic apparatus 100, the controller 18 executes composite image display processing in advance. The composite image display processing of the present embodiment is the same as the composite image display processing of the first embodiment, but the content of the composite image data is different. The controller 18 causes the display part 17 to display composite image data of a composite image of the generated ultrasound image data and an illustration image of illustration image data generated in illustration image data generation processing to be described later.

The illustration image data generation processing will be described focusing on differences from the first optical image setting process in FIG. 9. In the illustration image data-generating processing, step S11 to S14 of the first optical image setting process are common. In parallel with this, the controller 18 performs image analysis on the optical image data acquired in step S11 and detects the puncture needle 5. The controller 18 detects the position and the (insertion) direction of the puncture needle 5 in the optical image from the detected puncture needle 5. The controller 18 also analyzes the optical image of the optical image data to detect the position and direction of the ultrasound probe main body 21 in the optical image. The controller 18 determines the schematic diagram of the parallel method or the crossing method from the table of FIG. 6, based on the direction of the user in step S13, the detected position and direction of the puncture needle 5, and the detected position and direction of the ultrasound probe main body 21.

The controller 18 generates, for the determined schematic diagram, an illustration image data corresponding to the orientation of the optical image of the step S14. The illustration image of the image data includes a needle image corresponding to the detected position and direction of the puncture needle 5 and a probe image corresponding to the detected position and direction of the ultrasound probe main body 21. The controller 18 causes the image combining section 15 to generate composite image data of a composite image of the illustration image of the illustration image data and the ultrasound image of the ultrasound image data. At this time, if the determined schematic diagram corresponds to the crossing method, the controller 18 generates part data of a line 690 as the UI. The controller 18 causes the image combining section 15 to combine the parts data of the line 690 into the composite image data.

In the illustration image data generating process, similarly to the first optical image setting process, after the illustration image data is generated, the process proceeds to the first step (acquisition of first optical image data). Therefore, when the position of the user, the position and the direction of the puncture needle 5 and the ultrasound probe 2 change, the illustration image and the positions of the needle image and the probe image change (follow) in real time.

As described above, according to the present embodiment, the reference image displayed together with the ultrasound image is an illustration image indicating the positions and directions of the ultrasound probe 2 (ultrasound probe main body 21) and the puncture needle 5 in the optical image. The controller 18 performs image analysis on the optical image data to acquire the positions and orientations of the ultrasound probe 2 and the puncture needle 5. The controller 18 generates illustration image data of an illustration image indicating the positions and orientations of the ultrasound probe 2 and the puncture needle 5. Therefore, without using the optical image, the user can easily confirm the positional relationship and the direction of ultrasound probe 2 and puncture needle 5, and also confirm the puncture mode (the parallel method or the crossing method). Furthermore, since the illustration image is easy to see, the display size of the illustration image can be made smaller than that of the optical image. In this case, the display size of the ultrasound image can be increased, and the user can perform the puncture procedure more intuitively and easily.

Further, the controller 18 performs image analysis on the optical image data to acquire a puncture mode (parallel method or crossing method) of puncture based on the positions and orientations of the ultrasound probe 2 and the puncture needle 5. The controller 18 sets a part (the line 690 in the parallel method) of the UI as a display element corresponding to the acquired puncture mode to be displayed together with the ultrasound image and the illustration image. Therefore, the parts of the UI to be superimposed and displayed on the optical image and the ultrasound image can be automatically changed in accordance with the puncture mode, thus improving user operability.

Note that the UI to be combined with the composite image is not limited to the line 690 according to the parallel method or the crossing method. For example, as shown in FIG. 15, a composite image 700 may be displayed. The composite image 700 is a composite image in a case where the parallel method is determined. The composite image 700 includes an ultrasound image 710, an optical image 720 instead of an illustration image, and a needle trajectory prediction line 730 as a UI. The needle trajectory prediction line 730 is a linear part indicating a predicted path of insertion of the puncture needle 5 in the parallel method. Among the three lines of the needle locus prediction line 730, the middle line is the center line (locus on the extension of the puncture needle 5). Two lines above and below the middle line indicate the width of the range in which the puncture needle 5 is assumed to move. If the parallel method is determined, the controller 18 analyzes the optical image data, predicts a predicted insertion path of the puncture needle 5, and generates part data on the needle trajectory prediction line 730 corresponding to the prediction result. The controller 18 causes the image combining section 15 to combine the part data of the needle trajectory prediction line 730 with the composite image data.

Note that the calculation of the predicted insertion route of the puncture needle 5 is not limited to the prediction by only the analysis of the optical image data of the optical camera 31. In the prediction of the predicted route, a stereo camera system, a time of flight (TOF) sensor, a millimeter wave sensor, light detection and ranging (LiDAR), or the like may be used, or these may be used in combination. The stereo camera method is a measurement method using two optical cameras capable of measuring the depth direction of the puncture needle 5.

Further, as shown in FIG. 16, a composite image 740 may be displayed. The composite image 740 is a composite image in a case where the crossing method is determined. The composite image 740 includes an ultrasound image 750, an optical image 760 instead of an illustration image, and a mark 770 as a UI. The mark 770 is a rectangular part indicating a predicted insertion path of the puncture needle 5 in the crossing method. If the crossing method is determined, the controller 18 analyzes the optical image data to predict a predicted insertion path of the puncture needle 5, and generates part data on the mark 770 corresponding to the prediction result. The controller 18 causes the image combining section 15 to combine the parts data of the mark 770 with the composite image data.

### Fourth Embodiment

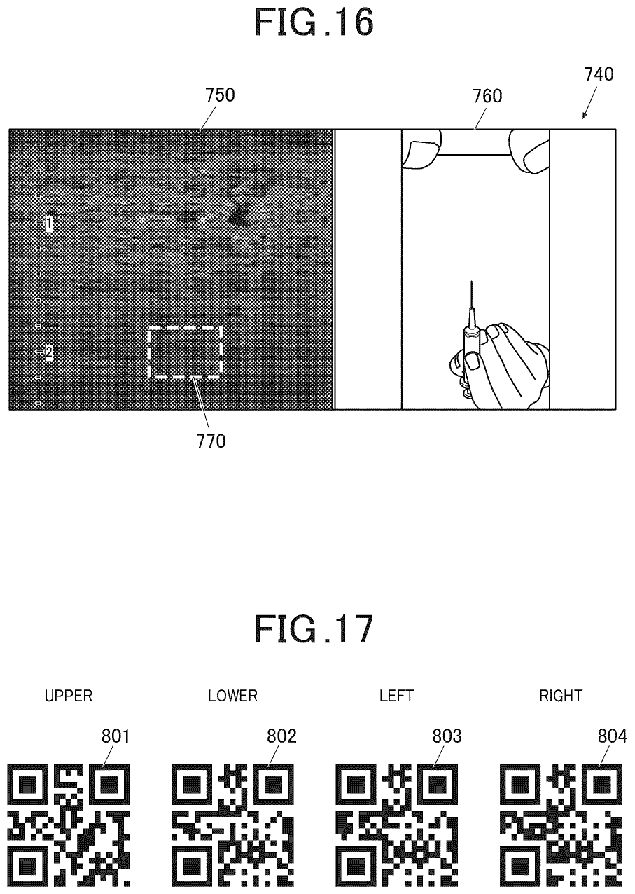

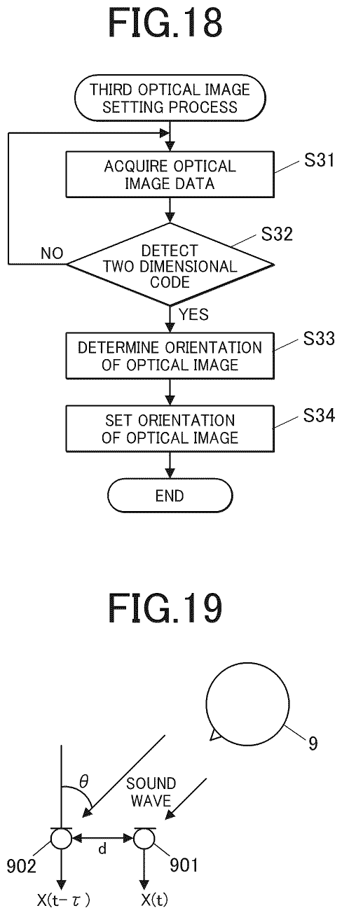

A fourth embodiment of the present invention will be described with reference to FIGS. 17 and 18. FIG. 17 is a diagram illustrating two dimensional codes 801, 802, 803, and 804. FIG. 18 is a flowchart illustrating the third optical image setting process.

In the first embodiment, the image analysis is performed on the optical image data of the optical camera 31, and the orientation of the optical image to be displayed is set to the appropriate direction. In the present embodiment, the user sets the orientation of the optical image to be displayed by reading a two dimensional code.

Also in the present embodiment, similarly to the first embodiment, the ultrasonic diagnostic apparatus 100 is used. However, in the ultrasonic diagnostic apparatus 100 of the present embodiment, the description of the configuration of the same parts as those of the ultrasonic diagnostic apparatus 100 of the first embodiment will be omitted, and different parts will be mainly described.

The ROM of the controller 18 stores, in place of the first optical image setting program, a third optical image setting program for executing third optical image setting process described later.

Next, a two-dimensional code to be read by the optical camera 31 of the ultrasonic diagnostic apparatus 100 will be described with reference to FIG. 17. As illustrated in FIG. 17, reading target are four types of two dimensional codes 801, 802, 803, and 804. Two dimensional codes 801, 802, 803, and 804 are quick response (QR) codes (R), and include different identification information. However, the target to be read is not limited to the two dimensional code, and may be another type of two dimensional code or a symbol such as a barcode.

The terms “up,” “down,” “left,” and “right” above the two dimensional codes 801 to 804 indicate, for example, the position of the puncture needle 5 in the optical image. That is, according to the table of the schematic diagram of FIG. 6, the two dimensional code 801 includes the identification information of the pattern 04 in which the position of the puncture needle 5 is on the upper side. Provided that the insertion direction of the puncture needle 5 is downward.

Similarly, the two dimensional code 802 includes identification information of the pattern 03 in which the position of the puncture needle 5 is on the lower side. Two dimensional code 803 includes identification information of the pattern 02 in which the position of the puncture needle 5 is on the left. Two dimensional code 804 includes identification information of the pattern 01 in which the position of the puncture needle 5 is on the right side.

Two dimensional codes 801 to 804 are printed on, for example, a sheet. The user holds a sheet on which two dimensional codes 801 to 804 are printed. Alternatively, the two dimensional codes 801 to 804 may be displayed on a part of the display screen of the display part 17.

Next, with reference to FIG. 18, operation of the ultrasonic diagnostic apparatus 100 will be described. In advance, as in the first embodiment, the controller 18 executes the composite image display processing. The user inserts the puncture needle 5 into the target site of the subject by the parallel method or the crossing method while visually checking the ultrasound image and the optical image of the composite image.

The third optical image setting process is processing for setting the orientation of the composite image of the optical image data generated in the above-described composite image processing to an appropriate direction on the basis of the two dimensional code reading result. For example, triggered by the execution of the composite image display processing, the controller 18 executes the third optical image setting process in accordance with the second optical image setting program in the ROM.

The user views the optical image of the composite image being displayed on the display part 17. When the user wants to change the orientation of the optical image so that the position of the user in the optical image is downward, the user moves the optical camera 31 on the ultrasound probe main body 21. The user causes the moved optical camera 31 to read the sheet or one of the two dimensional codes 801 to 804 being displayed.

First, the controller 18 acquires the generated optical image data from the optical image generator 141 (step S31). The controller 18 decodes the two dimensional code in the optical image data acquired in step S31, and determines whether (the pattern information of) the two-dimensional code has been detected (step S32). When the two dimensional code is not detected (step S32; NO), the process proceeds to step S31. When the two dimensional code is detected (step S32; YES), the controller 18 determines the appropriate orientation of the optical image of the user viewpoint with respect to the current optical image based on the pattern information of the detected two dimensional code (step S33). For example, the controller 18 analyzes the optical image to detect the position (direction) of the puncture needle 5. The controller 18 determines the orientation of the optical image such that the position of the puncture needle 5 corresponds to the pattern information and the position of the user corresponding to the position of the puncture needle 5 is located on the lower side. The controller 18 sets the orientation of the optical image of the optical image data to the direction determined in step S33 (step S34). Thereafter, the processing proceeds to step S31.

As described above, according to the present embodiment, the controller 18 detects the position of the puncture needle 5 in the optical image based on the pattern information obtained by decoding the two dimensional code included in the optical image of the optical image data. The controller 18 sets the orientation of the optical image to be displayed such that the position of the user corresponding to the detected position of the puncture needle 5 is located on the lower side. Therefore, when the user optically images the two dimensional code of the desired pattern information, the orientation of the optical image to be displayed together with the ultrasound image of the puncture can be easily and appropriately set so that the position of the user is on the lower side. Therefore, the user can intuitively and easily perform a puncture procedure.

### First Modification Example

A first modification example of the fourth embodiment described above will be described. The third embodiment described above has the configuration in which the two dimensional code of the optical image is read to acquire the pattern information, and the orientation of the optical image is determined and changed. The present modification example is configured to recognize a voice uttered by the user to acquire pattern information, and to determine and change the orientation of the optical image.

Also in the present modification example, the ultrasonic diagnostic apparatus 100 is used as an apparatus configuration. However, the ultrasonic diagnostic apparatus main body 1 includes a sound input section (not shown) connected to the controller 18. The sound input section is a microphone, receives input of a user's voice, and outputs a voice signal thereof to the controller 18. The speech to be recognized is “pattern 01”, “pattern 02”, “pattern 03”, or “pattern 04”. However, the speech to be recognized is not limited to the above example as long as it corresponds to each piece of pattern information.

Next, operation of the ultrasonic diagnostic apparatus 100 will be described. In advance, as in the first embodiment, the controller 18 executes the composite image display processing. The user inserts the puncture needle 5 into the target site of the subject by the parallel method or the crossing method while visually checking the ultrasound image and the optical image of the composite image.

The controller 18 executes optical image setting process similar to the third optical image setting process. In step S32, the controller 18 performs voice recognition on the voice data via the sound input section, and determines whether pattern information or corresponding voice information has been detected. When the audio information is detected (step S32; YES), in step S33, the controller 18 determines the appropriate orientation of the optical image of the user viewpoint with respect to the current optical image based on the pattern information of the detected audio information.

As described above, according to the present modification example, the user utters the voice of the desired pattern information, and thus it is possible to easily and appropriately set the orientation of the optical image to be displayed together with the ultrasound image of the puncture such that the position of the user is on the lower side.

### Second Modification Example

Referring to FIG. 19, a second modification example of the third embodiment described above will be described. FIG. 19 is a diagram illustrating the user 9 and the sound input sections 901 and 902.

The fourth embodiment described above has the configuration in which the two dimensional code of the optical image is read to acquire the pattern information, and the orientation of the optical image is determined and changed. In this modification example, the direction of an object such as the ultrasound probe main body 21 is detected by the sound input section, and the orientation of the optical image is determined and changed.

Also in the present modification example, the ultrasonic diagnostic apparatus 100 is used as an apparatus configuration. Provided that the ultrasonic diagnostic apparatus 100 includes sound input sections 901 and 902 connected to the controller 18 via cables. The sound input sections 901 and 902 are microphones. The sound input sections 901 and 902 receive an input of a sound wave from the user 9 and output a sound signal thereof to the controller 18. The sound input sections 901 and 902 are arranged on, for example, the ultrasound probe 2 or the housing of the ultrasonic diagnostic apparatus main body 1.

As illustrated in FIG. 19, a case where the user 9 is located and the sound input sections 901 and 902 are arranged is considered. The sound input sections 901 and 902 are arranged at a distance d. The axis perpendicular to the axis of the distance d and the sound wave direction of the voice uttered by the user 9 intersect each other at an angle θ. In such a case, the audio signal of the sound input section 901 is represented by X (t). An audio signal of the sound input section 902 is represented by X (t−τ). T is time. T is a time difference. The θ is expressed by the following Expression (1). θ=sin⁻¹ (c τ/d) . . . (1) Here, c is the speed of sound.

Therefore, by analyzing the time difference t of the voice signals, the direction from the user 9 to the sound input sections 901 and 902 can be calculated. For example, when the sound input sections 901 and 902 are arranged side by side in the long axis direction of the ultrasound probe main body 21, the orientation of the ultrasound probe main body 21 (optical camera 31) on a plane from the user 9 can be known.

In the first optical image setting process, for example, in the detection of the direction of the user in step S13, the controller 18 may use information on an analysis result of sound signals input from the sound input sections 901 and 902. The information on the analysis result of the sound signal is, for example, the direction of the user 9 from the ultrasound probe main body 21 (optical camera 31). Furthermore, in the third embodiment described above, it is conceivable to use the direction of the user 9 from the ultrasound probe main body 21 for the generation of the illustration image data.

In the above description, as the computer-readable media of the program according to the present invention, an example of using a ROM has been disclosed, but the present invention is not limited to this example. As other computer-readable media, a nonvolatile memory such as a flash memory and a portable recording medium such as a CD-ROM can be applied. As a medium for providing data of the program according to the present invention via a communication line, a carrier wave is also applied to the present invention.

Note that the description in the above embodiment and modification example is an example of the ultrasonic diagnostic apparatus, the image display method, and the program according to the present invention, and the present invention is not limited to this. For example, at least two of the above-described embodiment and modification example may be combined as appropriate.

According to the embodiments, it is possible to easily and appropriately change the orientation of the reference image to be displayed together with the ultrasound image of the puncture.

Although embodiments and modification examples of the present invention have been described and illustrated in detail, the disclosed embodiments and modification examples have been created for purposes of illustration and example only, and not limitation. The scope of the present invention should be interpreted by terms of the appended claims.

The entire disclosure of Japanese Patent Application No. 2024-079925 filed on May 16, 2024 is incorporated herein by reference in its entirety.

What is claimed is:

1. An ultrasonic diagnostic apparatus comprising:
an ultrasound image generator that generates ultrasound image data from reception signals of an ultrasound probe that transmits and receives ultrasound waves to and from a subject;
an optical image capturer that generates optical image data by optically imaging puncture into the subject using a puncture needle; and
a hardware processor that simultaneously displays an ultrasound image of the ultrasound image data and a reference image based on the optical image data, detects a position of a user in an optical image of the optical image data, and sets an orientation of the reference image to be displayed so that the detected position of the user is located on a lower side.

2. The ultrasonic diagnostic apparatus according to claim 1, wherein the hardware processor performs image analysis on the optical image data to detect the position of the user in the optical image.

3. The ultrasonic diagnostic apparatus according to claim 2, wherein
the optical image capturer includes a first optical image capturer that optically images puncture of the subject with the puncture needle to generate first optical image data, and a second optical image capturer that optically images puncture of the subject with the puncture needle in an imaging direction different from an imaging direction of the first optical image capturer to generate second optical image data, and
the hardware processor simultaneously displays an ultrasound image of the ultrasound image data and a reference image based on the first optical image data, and performs image analysis on at least one of the first optical image data and the second optical image data to detect the position of the user in a first optical image of the first optical image data.

4. The ultrasonic diagnostic apparatus according to claim 2, wherein the hardware processor detects a position of the puncture needle in the optical image based on pattern information obtained by decoding a symbol included in the optical image of the optical image data, and sets an orientation of the reference image to be displayed such that a position of the user corresponding to the detected position of the puncture needle is located on a lower side.

5. The ultrasonic diagnostic apparatus according to claim 1, wherein the reference image is the optical image.

6. The ultrasonic diagnostic apparatus according to claim 1, wherein the reference image is an illustration image indicating positions and directions of the ultrasound probe and the puncture needle in the optical image.

7. The ultrasonic diagnostic apparatus according to claim 6, wherein the hardware processor acquires the positions and the directions of the ultrasound probe and the puncture needle by performing image analysis on the optical image data, and generates illustration image data of an illustration image indicating positions and orientations of the ultrasound probe and the puncture needle.

8. The ultrasonic diagnostic apparatus according to claim 1, wherein the hardware processor performs image analysis on the optical image data to acquire a puncture mode of the puncture based on positions and directions of the ultrasound probe and the puncture needle, and sets a display element corresponding to the acquired puncture mode to be displayed together with the ultrasound image and the reference image.

9. An image display method comprising:
ultrasound image generating of generating ultrasound image data from reception signals of an ultrasound probe that transmits and receives ultrasound waves to and from a subject;
optical imaging of optically imaging puncture into the subject using a puncture needle to generate optical image data; and
controlling of simultaneously displaying an ultrasound image of the ultrasound image data and a reference image based on the optical image data, detecting a position of a user in an optical image of the optical image data, and setting an orientation of the reference image to be displayed such that the detected position of the user is located on a lower side.

10. A non-transitory recording medium storing a computer-readable program for a computer of an ultrasonic diagnostic apparatus comprising: an ultrasound image generator that generates ultrasound image data from reception signals of an ultrasound probe that transmits and receives ultrasound waves to and from a subject; and an optical image capturer that optically captures puncture into the subject using a puncture needle to generate optical image data, the program causing the computer to perform
controlling of simultaneously displaying an ultrasound image of the ultrasound image data and a reference image based on the optical image data, detecting a position of a user in an optical image of the optical image data, and setting an orientation of the reference image to be displayed such that the detected position of the user is located on a lower side.
